(19) United States
(12) Patent Application Publication
(10) Pub. No.: US 2022/0378714 A1
(43) Pub. Date: Dec. 1, 2022

(54) USE OF CANNABIDIOL PREPARATIONS IN THE TREATMENT OF FRAGILE X SYNDROME

(71) Applicant: GW Research Limited, Cambridge (GB)

(72) Inventors: Geoffrey GUY, Cambridge (GB); Volker KNAPPERTZ, Cambridge (GB); Benjamin WHALLEY, Cambridge (GB); Marie WOOLLEY-ROBERTS, Cambridge (GB)

(21) Appl. No.: 17/770,435

(22) PCT Filed: Oct. 23, 2020
(86) PCT No.: PCT/GB2020/052696

§ 371 Date: Apr. 20, 2022

(30) Foreign Application Priority Data
Oct. 25, 2019 (GB) 1915517.5

(51) Int. Cl. A61K 31/05; A61P 25/28; A61K 31/352; A61P 25/08; A61K 36/185

(52) U.S. Cl. CPC A61K 31/05; A61P 25/28; A61K 31/352; A61P 25/08; A61K 36/185; A61K 2236/17; A61K 2236/37

(57) ABSTRACT

The present invention relates to the use of a cannabidiol (CBD) preparation in the treatment of Fragile X syndrome (FXS). In particular the CBD preparation is characterized by chemical components and/or functional properties that distinguish them from prior CBD compositions. Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### FIELD OF THE INVENTION

The present invention relates to the use of a cannabidiol (CBD) preparation in the treatment of Fragile X syndrome (FXS). In particular the CBD preparation is characterized by chemical components and/or functional properties that distinguish them from prior CBD compositions.

Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### BACKGROUND TO THE INVENTION

Fragile X syndrome (FXS) co-occurs with autism in many cases and is the most common cause of inherited learning disability, occurring in 1 in 3,600 males and 1 in 8,000 females. FXS is caused by the presence of an apparently unstable or ‘fragile’ site located on the FMR1 gene on the X chromosome. The instability is caused by an excess of genetic code in this region. Males with FXS typically show mild to severe learning disability while females with FXS usually have a mild learning disability.

Recent studies of individuals with FXS show a fairly consistent pattern of association with ASD. The percentage of individuals with FXS showing ASD characteristics or meeting ASD criteria is up to 50%. Severe ASD is relatively rare in FXS and a milder presentation of ASD-like features is more characteristic.

Impairments in social interaction in FXS are characterized by social anxiety, extreme shyness and eye gaze avoidance. These characteristics are also observed in individuals with ASD. The social impairments associated with FXS often increase as the patient gets older.

The major symptom of FXS is intellectual disability with an average IQ of 40 in males who have complete silencing of the FMR1 gene. The main difficulties in individuals with FXS are with working and short-term memory, executive function, visual memory, visual-spatial relationships, and mathematics, with verbal abilities being relatively spared.

FXS sufferers also present with prominent characteristics which may include an elongated face, large or protruding ears, flat feet, larger testes (macro-orchidism), and low muscle tone.

FXS patients also suffer from recurrent middle ear infection and sinusitis. Speech may be cluttered or nervous. Behavioural characteristics may include stereotypic movements such as hand-flapping and atypical social development, particularly shyness, limited eye contact, memory problems, and difficulty with face encoding. These features mean that individuals with FXS also meet the diagnostic criteria for autism. Genetic mouse models of FXS have also been shown to have autistic-like behaviours.

Attention deficit hyperactivity disorder (ADHD) is found in the majority of males with FXS and 30% of females, making it the most common psychiatric diagnosis in those with FXS. Hyperactivity and disruptive behaviour peak in the preschool years and then gradually decline with age, although inattentive symptoms are generally lifelong.

From their 40s onward, males with FXS begin developing progressively more severe problems in performing tasks that require the central executive of working memory.

There is currently no drug treatment that has shown benefit specifically for FXS. However, medications are commonly used to treat symptoms of attention deficit and hyperactivity, anxiety, and aggression.

The present invention demonstrates an increased efficacy of a botanically derived purified CBD preparation which comprises minor amounts of the cannabinoids CBD-C1, CBDV, CBD-C4 and THC over a synthetic CBD which does not comprise minor amounts of cannabinoids in a mouse model of FXS. These data are particularly surprising particularly given the fact that the concentration of CBD within the botanically derived purified CBD preparation and the synthetic preparation were the same.

### BRIEF SUMMARY OF THE DISCLOSURE

In accordance with a first aspect of the present invention there is provided a cannabidiol (CBD) preparation for use in the treatment of Fragile X syndrome (FXS).

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the treatment of FXS is the treatment of seizures associated with FXS.

Alternatively, the treatment of FXS is the treatment of cognitive symptoms associated with FXS.

Alternatively, the treatment of FXS is the treatment of behavior associated with FXS.

Preferably the CBD is present is isolated from cannabis plant material.

More preferably at least a portion of at least one of the cannabinoids present in the CBD preparation is isolated from cannabis plant material.

Alternatively, the CBD is present as a synthetic preparation. In a further embodiment at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably the dose of CBD is 50 mg/kg/day.

In accordance with a second aspect of the present invention there is provided a method of treating Fragile X syndrome (FXS) comprising administering a cannabidiol (CBD) preparation to the subject in need thereof.

### BRIEF DESCRIPTION OF THE DRAWINGS

FIG. 1 shows the drug product manufacturing process.

FIG. 2 shows the total distance travelled in open field by Fmr1K and KO mice treated with botanically derived purified CBD and synthetic CBD.

FIG. 3 shows the rearing activity in open field by Fmr1K and KO mice treated with botanically derived purified CBD and synthetic CBD.

FIG. 4 shows the percentage discrimination (time spent in chamber) by Fmr1K and KO mice treated with botanically derived purified CBD and synthetic CBD.

FIG. 5 shows the percentage discrimination (interaction frequency) by Fmr1K and KO mice treated with botanically derived purified CBD and synthetic CBD.

FIG. 6 shows the total time spent by the WT and Fmr1 KO mice in the (A) partner mouse chamber; (B) centre chamber and (C) object chamber by Fmr1K and KO mice treated with botanically derived purified CBD and synthetic CBD.

FIG. 7 shows the effects of the test compounds on latency to onset of seizure by Fmr1K and KO mice treated with botanically derived purified CBD and synthetic CBD.

FIG. 8 shows the effects of the test compounds on median seizure score by Fmr1K and KO mice treated with botanically derived purified CBD and synthetic CBD.

FIG. 9 shows the summary seizure scores of Fmr1K and KO mice treated with botanically derived purified CBD and synthetic CBD.

### DEFINITIONS

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

The human equivalent dose (HED) can be estimated using the following formula:

$$HED = Animal dose ( mg / kg ) multiplied by \frac{Animal Km}{Human Km}$$

The Km for a mouse is 3 and the Km for a human is 37.

Thus, for a human a 100 mg/kg dose in a mouse equates to a human dose of about 8.1 mg/kg.

### DETAILED DESCRIPTION

### Preparation of Botanically Derived Purified CBD

The following describes the production of the botanically derived purified CBD which comprises greater than or equal to 98% w/w CBD and less than or equal to other cannabinoids was used in Example 1 below.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table A and the CBD API is described in Table B.

TABLE A
CBD botanical raw material specification

| Test | Method | Specification |
|---|---|---|
| Identification: | | |
| A | Visual | Complies |
| B | TLC | Corresponds to standard |
| | | (for CBD & CBDA) |
| C | HPLC/UV | Positive for CBDA |
| Assay: | | |
| CBDA + CBD | In-house | NLT 90% of assayed |
| | (HPLC/UV) | cannabinoids by peak area |
| Loss on Drying | Ph. Eur. | NMT 15% |
| Aflatoxin | UKAS method | NMT 4 ppb |
| Microbial: | | |
| TVC | Ph. Eur. | NMT10⁷ cfu/g |
| Fungi | | NMT10⁵ cfu/g |
| E. coli | | NMT10² cfu/g |
| Foreign Matter: | Ph. Eur. | NMT 2% |
| Residual Herbicides and | Ph. Eur. | Complies |
| Pesticides | | |

TABLE B
Specification of an exemplary botanically
derived purified CBD preparation

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass spectrum |
| | | of major peak corresponds to |
| | | certified CBD Reference Standard |
| Identification C | FT-IR | Conforms to reference spectrum |
| | | for certified CBD Reference |
| | | Standard |
| Identification D | Melting Point | 65-67° C. |
| Identification E | Specific Optical | Conforms with certified CBD |
| | Rotation | Reference Standard; −110° |
| | | to −140° (in 95% ethanol) |
| Total Purity | Calculation | ≥98.0% |
| Chromatographic | HPLC-UV | ≥98.0% |
| Purity 1 | | |
| Chromatographic | GC-FID/MS | ≥98.0% |
| Purity 2 | | |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual | | |
| Solvents: | | |
| Alkane | GC | NMT 0.5% w/w |
| Ethanol | | NMT 0.5% w/w |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:

a. Growing
b. Direct drying
c. Decarboxylation
d. Extraction—using liquid CO2
e. Winterization using ethanol
f. Filtration
g. Evaporation

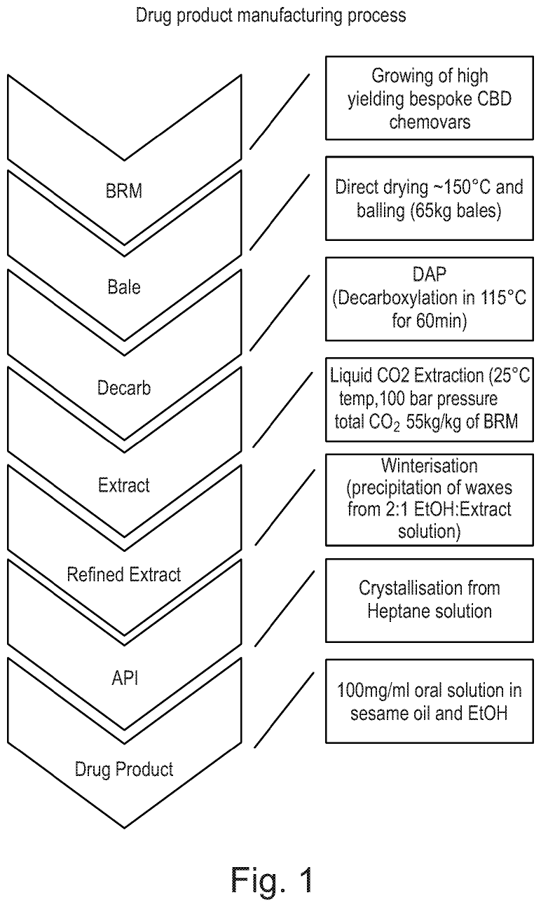

The process is described in greater detail below and in FIG. 1. High CBD yielding chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ at a temperature of 25° C. and 100 bar to produce botanical drug substance. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:

a. Crystallization using C5-C12 straight chain or branched alkane
b. Filtration
c. Vacuum drying

The BDS produced using the methodology above was dispersed in C5-C12 straight chain or branched alkane. The mixture was manually agitated to break up any lumps and the sealed container then placed in a freezer for approximately 48 hours. The crystals were isolated via vacuum filtration, washed with aliquots of cold C5-C12 straight chain or branched alkane, in this case heptane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in a mouse model of Fragile X syndrome.

### EXAMPLE 1: CANNABIDIOL (CBD) IN A MOUSE MODEL OF FRAGILE X SYNDROME

This Example demonstrates the effect of botanically derived purified CBD and synthetic CBD on mice in a mouse model of Fragile X syndrome (FXS).

### Methods

### Animals

Male Fmr1 KO and wild type (WT) mice were bred by crossing homozygous females stock 004624, FVB.129P2-Fmr1tm1Cgr/J with WT males. A total of 60 male mice were used for behavioural testing in this study: 10 WT and 50 Fmr1 KO.

The 50 male Fmr1 KO mice were allocated to the following treatment groups:

a) veh

b) CBD (Syn) 20 mg/kg i.p.

c) CBD (Syn) 200 mg/kg i.p.

d) CBD (BOT0.08%) 20 mg/kg i.p.

e) CBD (BOT-0.08%) 200 mg/kg i.p.

During the course of the study animals were maintained on a 12/12 light/dark cycle and the room temperature was maintained between 20 and 23° C. with a relative humidity maintained around 50%. Chow and water were provided ad libitum for the duration of the study.

### Compound Formulation

Test compounds were evaluated at 2 doses each (20 and 200 mg/kg). Test compounds were injected i.p. (intraperitoneal) acutely at 60 min pre-treatment for audiogenic seizures (cohort 1). For all other behavioural testing, a separate cohort of animals were dosed i.p. once per day for 2 weeks prior to the start of testing and then for 2 weeks during testing, with 60 min pre-treatment on test days (cohort 2).

Test compounds were:
a) CDB (SYN)=synthetic CBD (0% THC)
b) CBD (BOT 0.08%)=botanically derived purified CBD (>98% purity) containing 0.08% THC

Both compounds were formulated in ethanol: Kolliphor® HS15: 0.9% saline (2:1:17) and were added in the same order into opaque glass bottles.

### Body Weight

Body weight was measured prior to the test in the audiogenic seizure cohort. For the behavioral testing cohort, mice were balanced based on their body weights prior to the study and were measured twice a week during the course of the study.

### Audiogenic Seizures (Cohort 1)

### Dosing Regimen:

48 male mice were tested at 3 weeks of age. CBD (SYN OR BOT 0.08%) were tested at 2 doses each (20 and 200 mg/kg) following acute intraperitoneal administration at a dose volume of 10 ml/kg. Compounds were administered 60 minutes prior to test.

| | | |
|---|---|---|
| a) | Fmr1 KO veh | n = 10 |
| b) | Fmr1 KO CBD (Syn) 20 mg/kg i.p. | n = 10 |
| c) | Fmr1 KO CBD (Syn) 200 mg/kg i.p. | n = 10 |
| d) | Fmr1 KO CBD (BOT-0.08%) 20 mg/kg i.p | n = 10 |
| e) | Fmr1 KO CBD (BOT-0.08%) 200 mg/kg i.p. | n = 8 |

### Audiogenic Seizures:

Following pre-treatment with vehicle or test compound, mice were individually placed in a Plexiglas chamber and allowed to explore for 15 s following which they were exposed to a 125 dB tone for 2 minutes, followed by 1 minute of no sound, and then a further 2 minute tone. The mice were scored based on their response, latency, and seizure intensity:

0: no response
1: wild running and jumping
2: clonic seizures
3: clonic-tonic seizures
4: tonic seizures
5: respiratory arrest

The following endpoints were reported:
1. Latency to seize: Time to first seizure (maximum 300 s). Animals that did not show any seizure activity during the trial were given a score of 300 s.
2. Latency to respiratory arrest: Time to death (maximum 300 s). Animals that survived received a score of 300 s.
3. Seizure Score: Maximum score animals obtained during the 5 minute test.

After completion of testing, plasma and brain samples were collected from all surviving mice.

### Behavioral Testing (Cohort 2)

### Dosing Regimen:

60 male mice were tested at 8 weeks of age. CBD (SYN OR BOT 0.08%) were evaluated at 2 doses each (20 and 200 mg/kg) following once daily intraperitoneal administration, at a dose volume of 10 ml/kg. Compounds were administered for 2 weeks prior to the start of testing and throughout testing.

On the test day, compounds were administered 60 minutes prior to test.

| | | |
|---|---|---|
| a) | WT veh | n = 10 |
| b) | Fmr1 KO veh | n = 10 |
| c) | Fmr1 KO CBD (Syn) 20 mg/kg i.p. | n = 10 |
| d) | Fmr1 KOCBD (Syn) 200 mg/kg i.p. | n = 10 |
| e) | Fmr1 KO CBD (BOT-0.08%) 20 mg/kg i.p. | n = 10 |
| f) | Fmr1 KO CBD (BOT-0.08%) 200 mg/kg i.p. | n = 10 |

### Open Field:

The open field chambers are Plexiglas square chambers (27.3×27.3×20.3 cm; Med Associates Inc., St Albans, Vt.) surrounded by infrared photo beams (16×16×16) to measure horizontal and vertical activity. On the day of testing, mice were brought to the activity experimental room for acclimation to the conditions at least 1 h prior to testing. After a 60 minute pretreatment time, mice were placed in the center of an open field activity box for a 60 minute test period. After the completion of the test, mice were placed back in their home cage.

The time courses for locomotor and rearing activities were presented in 5-minute bins during the 60 minute test. Total distance traveled, and numbers of rears were also summed during the 60 minute test.

### Y-Maze:

The Y-maze test is an acute, rapid test that provides a measure of working memory and exploratory behavior. It is based on the innate tendency of a mouse to explore novel rather than familiar environments: thus, when allowed to explore a 3-armed maze (‘Y-maze’) the subjects alternate their arm visits so that they avoid re-visiting the most recently explored arm. Rodents with compromised working memory function are less able to recall which areas they have recently visited therefore displaying decreased spontaneous alternation. Global activity is reflected in the total number of visits to the different arms, while an alternation is defined as a visit in order to all three arms without revisiting any arm. The percentage of alternations performed, relative to the total number possible given an animals' overall number of visits, provides a useful measure of working memory.

The arms of the Y-maze consist of corridor-like enclosures, of sufficient size for the test subject to freely move around and rear up. In the Y-maze, three arms of approximately equal length radiate outwards from a central area. Each mouse is allowed to explore the Y-maze for a single trial lasting 8 minutes. Percent alternation and total arm entries were measured.

### Social Interaction:

The three-chamber paradigm is used to assess social interest. The Plexiglas testing apparatus consists of two side chambers and a center chamber, each 42.5 cm×17.5 cm×23 cm. The outer side chambers have a secondary inner chamber of 10 cm×17.5 cm×23 cm for holding the object or partner mouse, which is separated from the rest of the chamber by a perforated Plexiglas partition. Mice were brought to the experimental room for acclimation to the room conditions at least 1 hour prior to testing.

Group-housed naïve partner mice (age-matched C3H mice) were habituated to the chamber for 30 minutes the day before testing. Subject mice were habituated for 10 minutes in the center chamber of the apparatus, and then allowed to explore all three chambers for an additional 10 minutes. Post-habituation, the subject mice were restricted to the center chamber by the closure of two doorways. An inanimate object (pencil holder with a funnel top) was placed behind the partition in one side chamber and a partner mouse was placed behind the partition in the other side chamber of the apparatus. The doorways were then lifted and the subject was allowed to feely explore all three chambers for 10 min while movement was video-recorded and scored manually for sniffing of the mouse or object. The location of the inanimate object was alternated between side chambers for different subjects. Post-testing scoring was performed to determine frequency of visits per chamber, number of interactions and time spent in each compartment.

### Fear Conditioning:

On the first day of testing, mice were placed in a conditioning chamber to habituate to the context for 2 min after which a tone was presented for 20 s (CS). 30 s after the CS ended, a foot shock (1s, 0.5 mA) was presented (US). This pairing of the CS and US was repeated for a total of 3 times, with an interval of 60 s between pairings. Mice remained in the conditioning chamber for another 60 s and were then returned to their home cage.

On the second day, animals were tested for contextual memory where the mice were placed into the same chamber they were trained in, for a period of 5 minutes without shock or any other interference.

In the afternoon of the second day, animals were tested for cued memory ˜4 hours following contextual conditioning testing. Mice were placed in a novel context for 2 min (Pre-Cue). Then the CS was presented for a total of three times for 20 seconds, with 60 sec inter-trial intervals.

Freezing behavior, defined as the complete lack of movement during the 2 min pre-cue, 1 minute cue and 1 min post-cue as well as the 5 minute contextual FC, was captured automatically with a video system and FreezeView software (Coulbourn Instruments, PA, USA).

Terminal collections:

For the Audiogenic seizure cohort, plasma and brains were collected from all the surviving mice after the 5 minute test. For the behavioural testing cohort, mice were dosed once on the day following the last test day, and 60 minutes later plasma and brains were collected as described below:

Mice were decapitated and as much trunk blood as possible was collected into labelled microcentrifuge tubes (1.5 ml) containing Li Heparin and kept on ice for short term storage. Within 15 minutes the tubes were centrifuged at 40 C immediately for 10 minutes at 10,000-12,000 RPM in a refrigerated centrifuge. Plasma was extracted and 100 mg/mL L-ascorbic acid (2 g L-ascorbic acid dissolved in 20 ml de-ionized water, prepared fresh each day in non-transparent or amber glass vessel) was added accurately at a 1/1 (v/v) ratio before the sample was mixed thoroughly to stabilize the samples. The stabilized plasma was then pipetted into 40 μl aliquots, labelled, frozen and stored in the −80° C. freezer until shipment.

Whole brains were collected and weighed, then hemisected longitudinally. One brain half for bioanalysis was labelled and frozen on dry ice; the other half was dissected into the following brain regions (prefrontal cortex, hippocampus, cerebellum, brain stem) and stored in the −80° C. freezer until shipment.

### Statistical Analysis

Group sizes were selected based upon a priori power analysis to achieve 0.95 power and an alpha of 0.05 in experiments of primary interest with planned comparisons. In addition, many opportunistic measures were recorded, and hence it is likely that some of these opportunistic tests would not be sufficiently powered. Data for each variable was assessed for normality by D'Agostino & Pearson normality test and analyzed by one-way analysis of variance (ANOVA) if normally distributed, or appropriate non-parametric test (e.g. Kruskal-Wallis H test=one-way ANOVA on ranks) if data were not normally distributed, followed by planned comparisons; Dunn's for non-normally distributed data and Sidak's for normally distributed data (please refer to table below) corrected for multiple comparisons. An effect was considered significant if p<0.05. Data is represented as the mean+/−standard error of the mean (SEM) or as box whisker plots, as appropriate. Data were analyzed using Graphpad prism 8.2.0

### Results

### Behavioral Testing

### Body Weight

Mice were balanced across the different treatment groups by their body weight, hence no significant differences were observed (one-way ANOVA) in body weights among the groups prior to commencing the study.

Post hoc multiple comparisons indicated that treatment had no significant effect on the increase in body weight over time.

### Open Field

### Distance Traveled

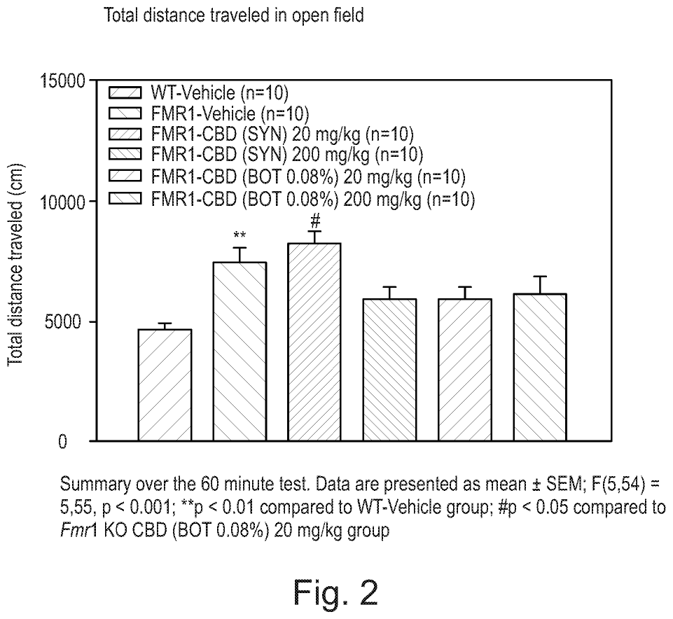

The effects of the test compounds on total distance traveled during the 60 minute test period are shown in FIG. 2. Post-hoc analysis revealed that Fmr1 KO-vehicle treated mice traveled significantly further (correlating with a hyperactive profile and confirming this phenotype in Fmr1 KO mice) compared to WT-vehicle treated mice, with no significant treatment effect detected between vehicle-treated and test compound-treated Fmr1 KO groups.

Mice treated with CBD (SYN) 20 mg/kg traveled significantly more than mice treated with CBD (BOT 0.08%) 20 mg/kg; although the latter is not significantly different from Fmr1 vehicle.

Two-way repeated measures ANOVA showed a significant main effect of treatment and time, but no interaction between the two factors. Post-hoc multiple comparisons indicated that Fmr1 KO-vehicle treated mice traveled significantly more than WT-vehicle treated mice during the first 25 min and during the 40 min-45 min period.

### Rearing Frequency

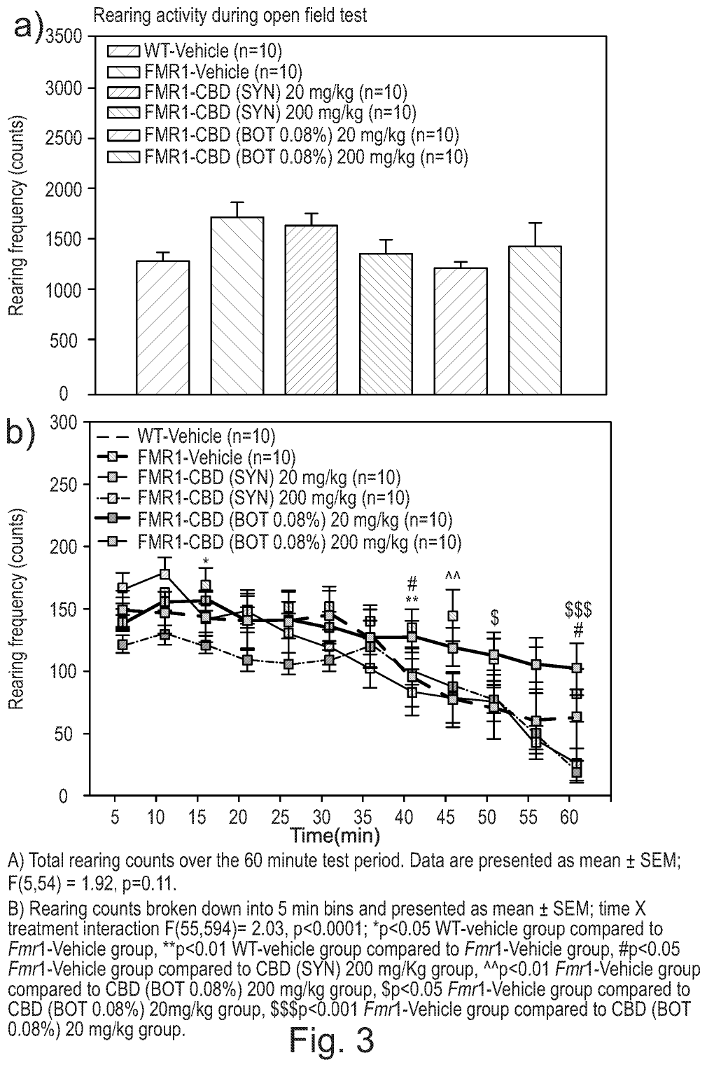

The effects of the test compounds on rearing frequency in the open field during the 60 minute test period are presented in FIG. 3A. One-way ANOVA did not reveal any significant treatment effects among any of the groups of mice.

The rearing frequency over the 60 min test period is split in to 5 min bins and is presented in FIG. 3B. Two-way repeated measures ANOVA, with treatment groups and time as factors, did not show a main effect of treatment, but revealed a significant interaction between the two factors.

Post-hoc multiple comparisons indicated that rearing counts in Fmr1 KO-vehicle group were significantly more compared to WT-vehicle treated group during the 10 min-15 min period and 35 min-40 min period. Moreover, Fmr1 KO-vehicle treated mice reared significantly more than the test compound treated groups as follows:

CBD (SYN) 200 mg/kg treated mice during minutes 35-40 and during minutes 55-60
CBD (BOT 0.08%) 200 mg/kg treated mice during minutes 40-45
CBD (BOT 0.08%) 20 mg/kg treated mice during minutes 45-50 and during minutes 55-60.

### Y-Maze

### Percent Spontaneous Alternation

The effect of the test compounds on percent alternations in the Y-maze did not reveal any significant treatment effects among any of the groups of mice when analyzed using one-way ANOVA (data not shown).

### Total Arm Entries

The total number of entries by mice to the three arms of the Y-maze did not reveal any significant treatment effects among any of the groups of mice when analyzed using one-way ANOVA (data not shown).

### Social Interaction

### Discrimination Index

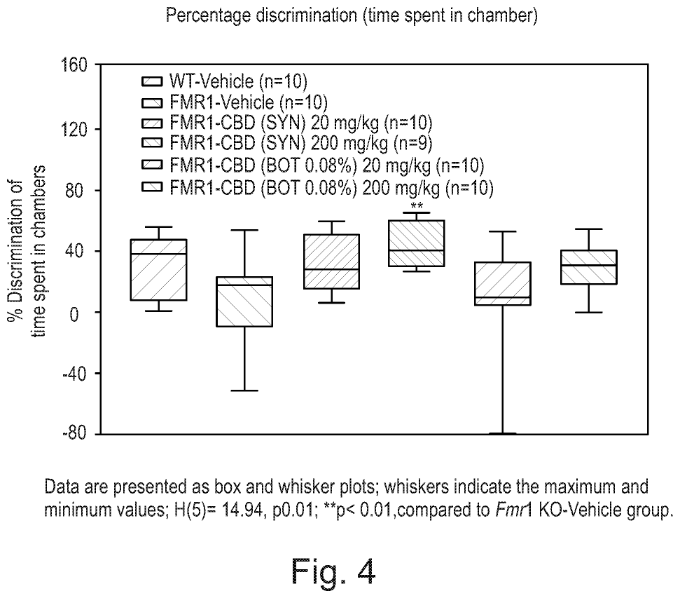

Discrimination percentage for time spent in chambers was calculated as (total time spent in mouse chamber-total time spent in object)/(total time spent in mouse+total time in object chamber)*100 and presented in FIG. 4.

Post hoc analysis, following significant Kruskal-Wallis test (H(5)=14.94, p=0.01), using Dunn's multiple comparison's test indicated that mice treated with the higher dose of CBD (SYN) showed enhanced discrimination compared to vehicle-treated Fmr1 KO mice. However, the Fmr1 vehicle mice were not statistically different from WT vehicle mice (p=0.41).

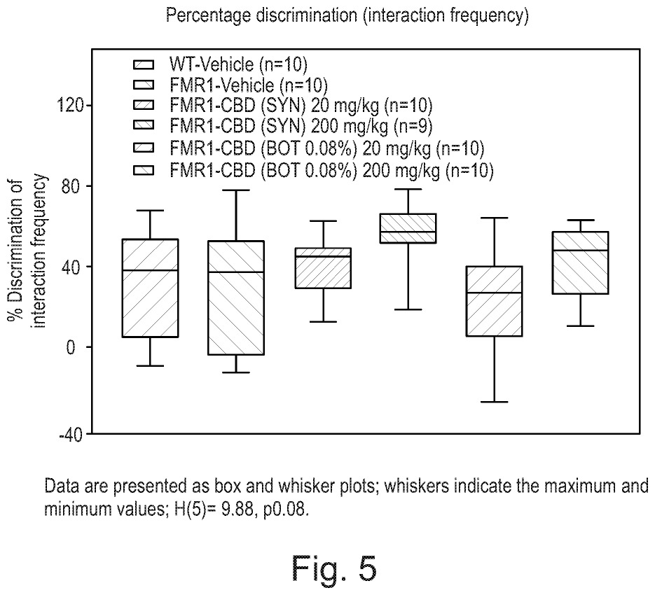

Discrimination percentage for interaction frequency was calculated as (number of interactions with mouse−number of interactions with object)/(total number of interactions with the mouse+the object)*100 and is presented in FIG. 5. No significant differences in discrimination between the mouse and the object were observed among any of the treatment groups (Kruskal-Wallis test; H(5)=9.88, p=0.08).

### Time Spent in Chambers

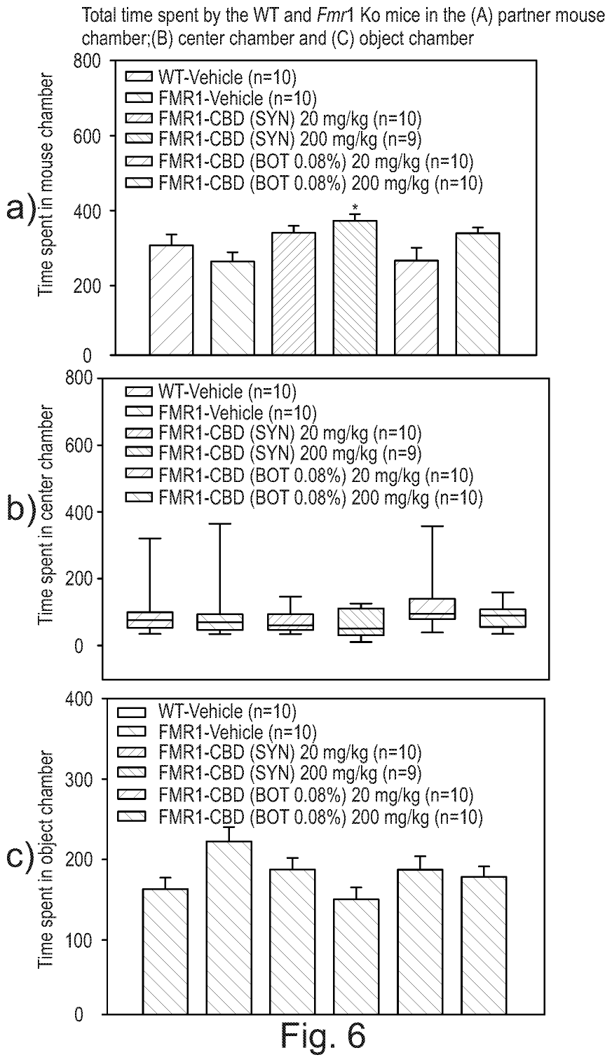

The effects of the test compounds on the total time spent in the partner mouse, center, and the inanimate object chambers by the WT and Fmr1 KO mice are presented in FIG. 6A-C.

Data were normally distributed for partner mouse and inanimate object times but not for time spent in the center. No significant genotype effect was observed between vehicle-treated groups in any of the three chambers. Mice treated with the higher dose of CBD (SYN) spent significantly more time in the partner mouse chamber, compared to Fmr1 KO vehicle—treated mice (FIG. 6A; one-way ANOVA, Sidak's multiple comparisons test).

There were no significant effects of treatment in the amount of time spent in the center (FIG. 6B; Kruskal-Wallis) or the object chamber (FIG. 6C; one-way ANOVA), among any of the treatment groups.

### Frequency of Visits Per Chamber

No difference was observed either between genotype or treatment groups by one-way ANOVA in the number of visits to the mouse chamber (data not shown).

A significant effect of genotype was observed in the frequency of visits to the object chamber in the vehicle-treated groups (one-way ANOVA followed by Sidak's multiple comparisons test). Fmr1 KO mice visited the object chamber more frequently compared to WT mice (data not shown).

### Frequency of Interactions

No significant effect of genotype or treatment was observed for the number of total interactions of WT and Fmr1 KO mice with the partner mouse and the inanimate object during the test (data not shown).

### Fear Conditioning

### Contextual Memory

No significant effect of treatment was seen on the effects of the test compounds on contextual memory, evaluated by the change in freezing behavior.

### Cued Memory

None of the compounds tested had any effect on the freezing behavior in animals during the pre-cue, cue, or the post-cue periods of the test (Kruskal-Wallis test).

### Audiogenic Seizure

### Body Weight

A separate cohort of animals at 3 weeks of age was used for the audiogenic seizure analysis. ANOVA did not reveal any significant differences in body weight among any of the treatment groups prior to the test. Notably, the vehicle response observed in this study was different from the typical response previously obtained when saline was used as the vehicle.

### Latency to Seizure

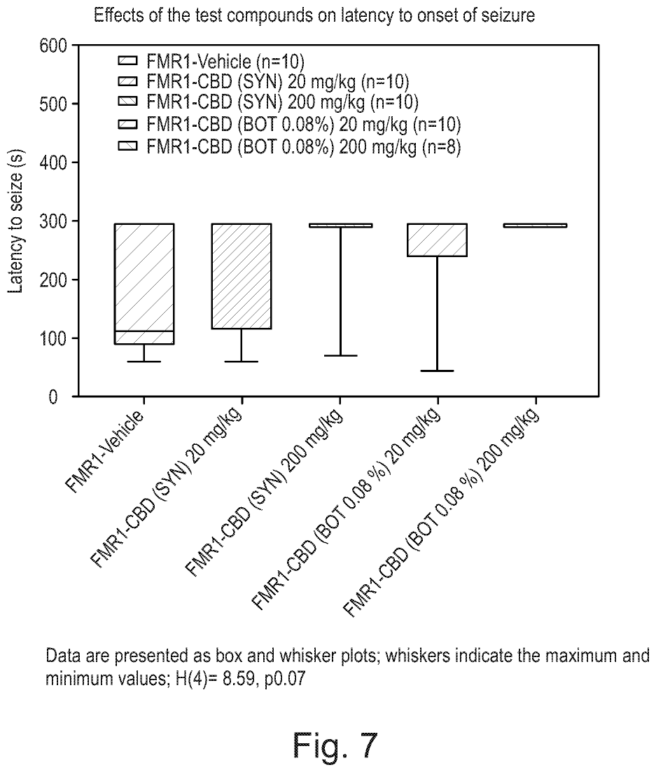

The effect of the test compounds on the latency to onset of seizure is shown in FIG. 7. A non-parametric one way ANOVA on ranks did not reveal any significant treatment effect among any of the treatment groups

### Seizure Score

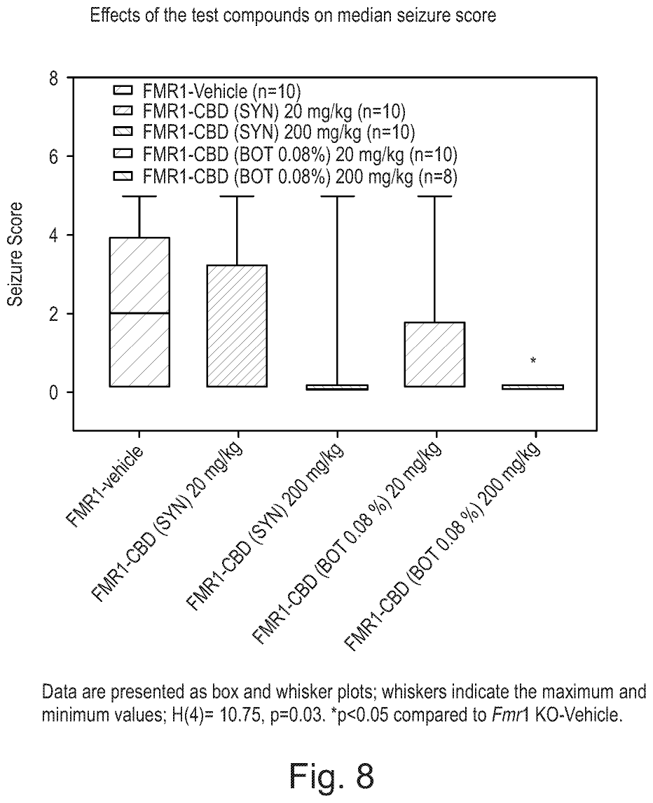

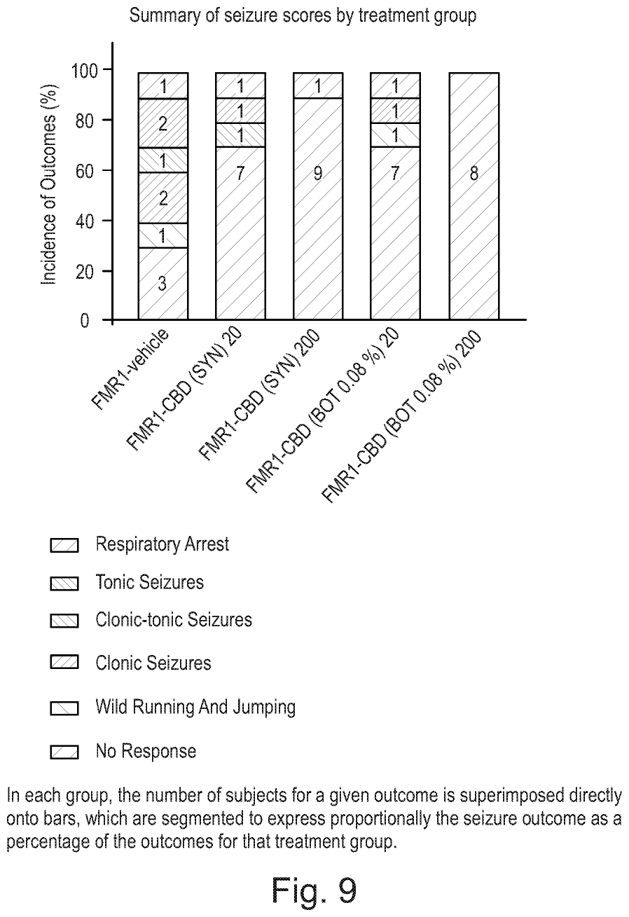

The effects of the test compounds on seizure score are presented in FIG. 8. Dunn's post-hoc multiple comparisons, following significant Kruskal-Wallis test (H(4)=10.74, p=0.03), identified a significant reduction in median seizure score in Fmr1 KO animals treated with CBD (BOT 0.08%), 200 mg/kg compared to vehicle-treated Fmr1 KO animals. Seizure outcomes by group are summarized in FIG. 9.

### CONCLUSION

The study evaluated the therapeutic efficacy of CBD (SYN or BOT 0.08%) at two doses each (20 and 200 mg/kg), in improving cognitive and other behavioral impairments in Fmr1 KO mice in addition to reducing seizures.

Neither the botanically derived purified CBD or the synthetic CBD were found to have any significant improvement on any of the social or memory related behaviors.

However, there was a significant reduction in median seizure score in Fmr1 KO animals treated with botanically derived CBD at 200 mg/kg compared to vehicle treated Fmr1 KO animals. This effect was not observed in the mice treated with synthetic CBD at 200 mg/kg suggesting there is an increased efficacy of the botanically derived purified CBD.

Such data are significant as they demonstrate that such a CBD composition may be useful in the treatment of epilepsy.

1. A method of treating Fragile X syndrome (FXS) comprising administering a cannabidiol (CBD) preparation to the subject in need thereof.

2. The method according to claim 1, wherein the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

3. The method according to claim 1, wherein the treatment of FXS is the treatment of seizures associated with FXS.

4. The method according to claim 1, wherein the treatment of FXS is the treatment of cognitive symptoms associated with FXS.

5. The method according to claim 1, wherein the treatment of FXS is the treatment of behavior associated with FXS.

6. The method according to claim 1, wherein the CBD is present is isolated from cannabis plant material.

7. The method according to claim 1, wherein at least a portion of at least one of the cannabinoids present in the CBD preparation is isolated from cannabis plant material.

8. The method according to claim 1, wherein the CBD is present as a synthetic preparation.

9. The method according to claim 8, wherein at least a portion of at least one of the cannabinoids present in the CBD preparation is prepared synthetically.

10. The method according to claim 1, wherein the dose of CBD is greater than 5 mg/kg/day.

11. The method according to claim 1, wherein the dose of CBD is 20 mg/kg/day.

12. The method according to claim 1, wherein the dose of CBD is 25 mg/kg/day.

13. The method according to claim 1, wherein the dose of CBD is 50 mg/kg/day.

14. (canceled)
